(19) United States
(12) Patent Application Publication
(10) Pub. No.: US 2009/0098102 A1
(43) Pub. Date: Apr. 16, 2009

(54) Cosmetic Composition Containing Enzyme and Amino Acid

(76) Inventors: Seong A. Cho, Gyeonggi-do (KR); Jun Cheol Cho, Gyeonggi-do (KR); Sang Hoon Han, Gyeonggi-do (KR)

Correspondence Address: NIXON & VANDERHYE, PC, 901 NORTH GLEBE ROAD, 11TH FLOOR, ARLINGTON, VA, 22203, US

(21) Appl. No.: 11/990,431

(22) PCT Filed: Mar. 17, 2006
(86) PCT No.: PCT/KR2006/000975

§ 371 Date: Apr. 18, 2008

(30) Foreign Application Priority Data
Aug. 18, 2005 (KR) 10-2005-0075473

(51) Int. Cl. A61K 8/66; A61Q 19/00

(52) U.S. Cl. 424/94.64; 424/94.63; 424/94.67

(57) ABSTRACT

The present invention relates to a skin cosmetic composition containing enzyme and amino acid. More particularly, the skin cosmetic composition according to the invention contains enzyme and amino acid, and thus safely improves stratum corneum thickening resulting from the progression of skin aging, improves skin drying occurring during keratin removal, and shows excellent skin moisturizing and whitening effects.

### TECHNICAL FIELD

The present invention relates to a skin cosmetic composition containing enzyme and amino acid. More particularly, the skin cosmetic composition according to the present invention contains enzyme and amino acid, and thus safely improves stratum corneum thickening (hyperkeratinization) resulting from the progression of skin aging, improves skin drying occurring during keratin removal, and shows excellent skin moisturizing and whitening effects.

### BACKGROUND ART

Generally, the skin is divided into three parts, starting from top to bottom: the epidermis (top skin layer), the dermis and the subcutaneous fat layer. The epidermis is divided into the stratum corneum, the stratum spinosum, the stratum granulosum, and the stratum basale, and the epidermal cells of the stratum basale are differentiated while moving upward toward the skin top layer and finally reach the stratum corneum. The epidermal cells which reached the stratum corneum lose nuclei and are filled with a water-insoluble protein, known as “keratin”, while these are converted into dead cells.

The stratum corneum consists of differentiated epidermal cells (keratinycoytes) and skin lipids filled therebetween, prevents the body's substances from going out of the body and performs a defense function to protect the human body from external physical, chemical and biological stimulus. Keratinocytes are connected through proteins, called “desmosomes”, and the desmosomes are degraded as they move toward the upper portion of the stratum corneum, so that the desmosomal adhesion between keratinocytes becomes weakened, and finally, the keratinocytes are separated and detached from the skin. In the case of normal skin, the stratum corneum consists of 15-20, and the time required for complete separation of these layers is 15-20 days (British Journal or Dermatology, 86, 14-19 1971).

In particular, aged skin, dry skin, acne skin and the like show the thickening of the stratum corneum, due to the separation of the stratum corneum which is late compared to the normal state, and the striking feature of the stratum corneum thickening, shown in the appearance of the skin, is the development of scales. The stratum corneum thickening leads is mainly attributable to a reduction in the moisturizing ability of the skin, the production of desmosome-degrading enzymes, a reduction in the activity of the enzyme, a reduction in cell activity and the like, and is caused by skin aging, UV exposure, environmental pollution and the like. When the stratum corneum thickened due to such internal and external factors becomes thin by an artificial method, the activity or regeneration of living cells underneath the stratum corneum will be increased so that scales shown in the appearance of the skin will be reduced, the skin becomes soft, and effects such as wrinkle removal and acne suppression and treatment will be obtained. Thus, many studies to solve the stratum corneum thickening have already been conducted. To solve the stratum corneum thickening, physically rubbing the skin or chemical peeling is used. Particularly, the chemical peeling has additional effects, such as the improvement of fine wrinkles, the improvement of rough skin, and the removal of fine spots. Substances used in the chemical peeling include trichloroacetic acid, phenol, alpha-hydroxy acid (AHA) and the like. Alpha-hydroxy acid used in the chemical peeling is used at a high concentration of at least 20-30%, but at a low concentration of less than 10%, it slowly exfoliates the stratum corneum (Journal of American Academy of Dermatology, 11, 867-879) and also shows effects such as an increase in skin moisturization and a reduction in fine wrinkles (Cutis, 43, 222-228). Thus, many products comprising alpha-hydroxy acid have recently been sold in the cosmetic or pharmaceutical industry. Also, it is known that the treatment and prevention of acne are possible by accelerating the peeling of keratin to remove keratin that makes face follicles narrow. Also, in skin whitening products, a keratin removal-accelerating substance for removing already produced melanin has been used together with a melanin production-inhibiting substance. However, because alpha-hydroxy acid has a shortcoming in that it causes stinging and irritation due to low pH, the control of the concentration and pH of alpha-hydroxy acid used is very important for the reduction of skin side effects. In attempts to overcome this shortcoming of alpha-hydroxy acid, studies to develop alpha-hydroxy acid derivatives or components having effects similar thereto, and studies to reduce the concentration of alpha-hydroxy acid used, are actively ongoing.

However, in the case of alpha-hydroxy acid derivatives, the consideration of formations for skin absorption is required because these derivatives have high molecular weight such that these cannot smoothly penetrate the skin. Also, in the case of keratin-softening components derived from plants such as pine apples or papayas, it is difficult to maintain the activity of the active ingredient in formulations, and thus it is difficult to expect the long-term effect of the active ingredient in general formulations. If the removal of keratin from the skin is operated using a keratin-softening component, skin drying also occurs to cause a reduction in the skin moisturizing ability, which is the fundamental cause of the stratum corneum thickening, or to develop skin scales or cause skin roughness, making the design of formulation difficult.

### DISCLOSURE

### Technical Problem

The present inventors have conducted studies to develop components which can effectively improve the stratum corneum thickening and also skin drying resulting from the stratum corneum thickening, and as a result, found that, when enzyme and amino acid are contained in a skin cosmetic composition, the cosmetic composition can accelerate the removal of keratin without irritating the skin and improve skin drying and skin roughness and the like, which are caused by the stratum corneum thickening, thereby completing the present invention.

Accordingly, it is an object of the present invention to provide a skin cosmetic composition which effectively improves the stratum corneum thickening without irritating the skin and, at the same time, maintains the content of moisture in the stratum corneum to improve skin roughness in the stratum corneum.

### Technical Solution

To achieve the above object, the present invention provides a cosmetic composition containing enzyme and amino acid.

Hereinafter, the present invention will be described in further detail.

The skin cosmetic composition according to the present invention contains enzyme and amino acid, and thus increases keratin removal effects and, at the same time, shows an excellent skin moisturizing effect by maintaining the moisture content of the stratum corneum even after the removal of keratin.

The enzyme, which is used in the present invention, is protease having a keratin removal effect, and specific examples thereof include serine protease, cysteine protease, metal protease and aspartic protease.

Meanwhile, examples of the amino acid, which is used in the present invention, include lysine, histidine, arginine, aspartic acid, threonine, serine, glutamic acid, proline, glycine, alanine, valine, methionine, isoleucine, leucine, tyrosine, phenylalanine, cystein, asparagine, glutamine, tryptophane, and derivatives thereof.

In the skin cosmetic composition according to the present invention, said enzyme is contained in an amount of 0.01-10 wt % based on the total weight of the composition. If the enzyme is used in an amount of less than 0.01 wt %, its effect will be insignificant, and if it used in an amount of more than 10 wt %, it will make it difficult to maintain skin safety and formulation stability.

Also, the amino acid is contained in an amount of 0.01-20 wt % based on the total weight of the composition. If the amino acid is used in an amount of less than 0.01 wt %, it will have little or no effect, and if it is used in an amount of more than 20 wt %, it will make it difficult to maintain skin safety and formulation stability.

The skin cosmetic composition according to the present invention has no particular limitation on its formulation. For example, the composition can be used as a formulation such as skin softener, skin lotion, massage cream, nutrient cream, pack, gel or stick-type cosmetic, or a transdermal formulation such as lotion, ointment, gel, cream, patch or spray.

### DESCRIPTION OF DRAWINGS

FIG. 1 shows measurement results for a change in melanin index in persons applied with compositions of Examples 1 to 3 of the present invention.

FIG. 2 shows measurement results for an increase in skin moisture content in persons applied with compositions of Examples 1 to 3 of the present invention.

### BEST MODE

Hereinafter, the present invention will be described in detail with reference to Examples and Experimental Examples. However, these examples are not to be construed to limit the scope of the present invention.

### Examples 1-3 and Comparative Examples 1-21

In Examples 1 to 3 and Comparative Examples 1 and 2, oil-in-water emulsions having compositions shown in Table 1 were prepared.

TABLE 1

| | | | | Comparative | Comparative |
|---|---|---|---|---|---|
| Component | Example 1 | Example 2 | Example 3 | Example 1 | Example 2 |
| Cetostearyl alcohol | 1.8 | 1.8 | 1.8 | 1.8 | 1.8 |
| Glyceryl monostearate | 1.5 | 1.5 | 1.5 | 1.5 | 1.5 |
| Stearic acid | 1.2 | 1.2 | 1.2 | 1.2 | 1.2 |
| Vegetable squalane | 10 | 10 | 10 | 10 | 10 |
| Propylene glycol monostearate | 1 | 1 | 1 | 1 | 1 |
| Polyoxyethylene cetylester | 0.3 | 0.3 | 0.3 | 0.3 | 0.3 |
| Preservative and antioxidant | qs | qs | qs | qs | qs |
| Carbomer | 0.13 | 0.13 | 0.13 | 0.13 | 0.13 |
| Glycerin | 4 | 4 | 4 | 4 | 4 |
| Triethylamine | 0.12 | 0.12 | 0.12 | 0.12 | 0.12 |
| Serine protease | 2.0 | 2.0 | 2.0 | — | 2.0 |
| Serine | 1.0 | 1.0 | — | — | — |
| Glutamic acid | 1.0 | — | 1.0 | — | — |
| Glycine | — | 1.0 | 1.0 | — | — |
| Purified water | To 100 | To 100 | To 100 | To 100 | To 100 |

### Experimental Example 1

### Measurement of Keratin Removal Effect in Human Body

The effects of Examples 1 to 3 and Comparative Examples 1 and 2 on the acceleration of keratin removal, shown when applied to the human body, were measured in the following manner.

Twenty 20-30-year-old healthy women as test subjects were on standby in constant temperature and constant humidity conditions (241° C. temperature and 40-50% humidity) for a given time after washing the forearms clean. Before the application of the samples, the color of the inner side of the upper arm of each subject was measured with a chromameter (CR-200, Minolta). Then, the color of a portion colored in brown with dihydroxy acetone (DHA) was measured and compared to the color measured before the application of the samples. Then, while the samples were applied to the subjects two times one day, the degree of discoloration was measured daily with the chromameter to determine the time taken for the skin to return to the original color (turn over time) was measured. The measurement results are shown in Table 2 below.

TABLE 2

| Samples | Turn over time (days) |
|---|---|
| Example 1 | 10.8 ± 1.8 |
| Example | 11.2 ± 2.2 |
| Example 3 | 12.6 ± 3.0 |
| Comparative Example 1 | 18.2 ± 3.3 |
| Comparative Example 2 | 14.4 ± 4.3 |

As can be seen from the results in Table 2, Examples 1-3 showed an improvement in keratin turnover, and particularly Examples 1 and 2 showed the highest improvement in keratin turnover.

### Experimental Example 2

### Examination of Change in Melanin Index in Human Body

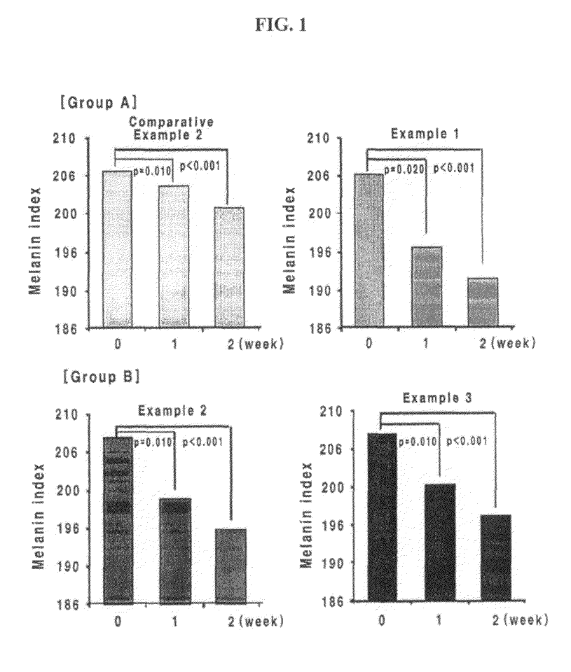

The melanin indexes of faces applied with the compositions of Examples 1-3 and Comparative Example 1 were measured with a Mexameter and compared with each other. Forty 20-30-year-old healthy women were divided into two groups of A and B, each consisting of 20 persons. In group A, Comparative Example 2 and Example 1 were applied on the left and right sides of the faces, and in group B, Examples 2 and 3 were applied. The measurement of melanin indexes for the applied faces was performed two times for 2 weeks. The measurement was performed for lesion sites (hyperpigmented sites) and non-lesion sites (lightly pigmented sites) on the left and right sides of the faces, and in the case of faces having no lesion, the most deeply pigmented site was used as the lesion site. Three measurements per one site were performed, the measured values were averaged, and a difference in melanin index before and after the application of the samples was determined. The melanin indexes were measured with Mexameter MX-18 (C+K, Germany), and based on the measurement results, the difference in melanin index before and after the application of the samples was statistically processed using Paired T-test (P=0.05). The measurement results are shown in FIG. 1.

As can be seen in FIG. 1, Examples 1-3 all showed an excellent effect of reducing melanin index, compared to Comparative Example 2. Particularly, Examples 1 and 2 showed a more excellent effect on the reduction of melanin index, and this is because, among the amino acids used, serine also showed the effect of beta-hydroxy acid.

### Experimental Example 3

### Measurement of Increase in Skin Moisture Content in Human Body

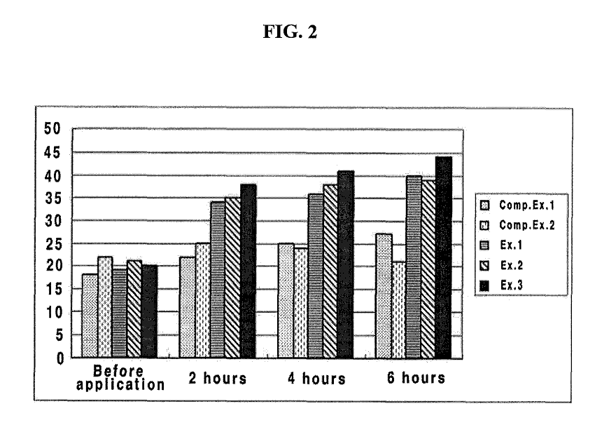

A change in skin moisture content, shown when the compositions of Examples 1-3 and Comparative Examples 1 and 2 were applied on the human body, was measured. Twenty 20-30-year-old women were on standby in constant temperature and constant humidity conditions (241° C. temperature and 40-50% humidity) for a given time after washing the forearms clean. After applying the samples on the forearms, the skin moisture content was measured. At 2, 4 and 6 hours after randomly applying the samples on the subjects, the skin moisture content was measured. The skin moisture content was measured with a corneometer (C+K AG., Germany). For comparison, the measured values were statistically processed using paired T-test in comparison with an untreated group. The measurement results are shown in FIG. 2.

As can be seen from the results shown in FIG. 2, although an increase in keratin removal effect generally involves a decrease in skin moisture content, the compositions of Examples 1-3 according to the present invention showed a significant increase in skin moisture content, and particularly Example 3 showed the highest increase. On the other hand, in the case of Comparative Example 2, containing only enzyme without amino acid, the skin moisture content was rather reduced, and this reduction is believed to occur in general keratin removal acceleration.

### INDUSTRIAL APPLICABILITY

As described above, the composition according to the present invention contains enzyme and amino acid, which have low irritation and high affinity to the skin, and thus it improves the hyperkeratinization of the skin, leading to improvements in keratin turnover time and skin brightness, and maintains skin moisture content and shows a skin moisturizing effect by improving skin surface drying which occurs in general keratin removal. Accordingly, the inventive composition is useful for skin external preparations for making the skin light and smooth.

1. A cosmetic composition for skin moisturization, which contains protease enzyme and amino acid.

2. A cosmetic composition for the removal of skin keratin, which contains protease enzyme and amino acid.

3. A cosmetic composition for skin whitening, which contains protease enzyme and amino acid.

4. The cosmetic composition of claim 1, wherein the enzyme is selected from the group consisting of serine protease, cysteine protease, metal protease and aspartic protease.

5. The cosmetic composition of claim 1, wherein the amino acid is selected from the group consisting of lysine, histidine, arginine, aspartic acid, threonine, serine, glutamic acid, proline, glycine, alanine, valine, methionine, isoleucine, leucine, tyrosine, phenylalanine, cystein, asparagine, glutamine, tryptophane, and derivatives thereof.

6. The cosmetic composition of claim 1, wherein the enzyme is contained in an amount of 0.01-10 wt % based on the total weight of the composition.

7. The cosmetic composition of claim 1, wherein the amino acid is contained in an amount of 0.01-20 wt % based on the total weight of the composition.
